(19) United States
(12) Patent Application Publication
(10) Pub. No.: US 2012/0101448 A1
(43) Pub. Date: Apr. 26, 2012

(54) Safe Disposable Injector with Changeable and Automatically Retractable Needle

(75) Inventor: Wenjun Jiang, Zhenjiang (CN)

(73) Assignee: WUXI YUSHOU MEDICAL APPLICANCE LTD., Wuxi (CN)

(21) Appl. No.: 13/380,463

(22) PCT Filed: Jul. 13, 2010
(86) PCT No.: PCT/CN10/01046

§ 371 Date: Dec. 22, 2011

(30) Foreign Application Priority Data
Jul. 15, 2009 (CN) 200910164711.1
Jul. 13, 2010 (CN) PCT/CN2010/001046

(51) Int. Cl. A61M 5/32

(52) U.S. Cl. 604/198

(57) ABSTRACT

A safe disposable injector with a changeable and automatically retractable needle includes a needle cylinder (1) and a plunger (2), and also includes a medicament-dispensing needle unit (3) and an injecting needle unit (4). The medicament-dispensing needle unit (3) and the injecting needle unit (4) are changed according to the requirement in use. The present invention will prevent harmful effects caused by needle deformation and variation of smoothness and sharpness during the injection to the human body. The operation of the present invention is the same as the convention injection operation. The present invention is convenient to use and reliable to operate. After injection, the injector needle can be retracted by an automatic retraction mechanism to avoid the accidental hurt to medical staff and patients by a polluted needle tip. The present invention is suitable for automatic production on the assembly line.

### FIELD OF INVENTION

The present invention relates to an injector, and more particular to a safe disposable injector, wherein the injecting needle is changeable and retractable after use.

### DESCRIPTION OF RELATED ARTS

It is common practice for a disposable syringe to administer injections and vaccinations since the disposable syringe is a one-time-use device for preventing infectious diseases and nosocomial cross-infection. Such disposable syringe, such as syringe with auto-destroy needle, syringe with manual retractable needle, or syringe with automatic retractable needle, are generally of the disposable nature and are normally discarded after a single use. However, the disposable syringe has complicated structural configuration and lacks of necessary operational function. For example, when preparing dry powder injection, the syringe is used for injecting solvent into a sealed bottle in order to dissolve the dry powder in liquid form. In particular, the needle must be penetrated through the bottle stopper in order to inject the solvent into the bottle. In other words, the needle will be bent or deformed during the penetration. In addition, the tip of the needle will be blunt and the smoothness of the needle body will be substantially reduced after the penetration. Therefore, the syringe must be discarded after the preparation of the dry powder injection since the needle cannot be re-used to administer injection for human body. In fact, the syringe is prohibited for being re-used after preparation of the dry powder injection in many countries. Therefore, the physician must use one disposable syringe to prepare the dry powder injection and another one to administer injection. Accordingly, there is no syringe with changeable needle in the market, especially for the retractable needle.

### SUMMARY OF THE PRESENT INVENTION

The invention is advantageous in that it provides a safety disposable injector, which solves the existing problems of the syringe, wherein the needle is changeable and automatically retractable after use. The present invention is convenient to use and reliable to operate. After injection, the injector needle can be retracted by an automatic retraction mechanism to avoid an injury by accidental needle sticks as serious health hazard and danger of exposure to fatal blood-transmitted viruses.

Additional advantages and features of the invention will become apparent from the description which follows, and may be realized by means of the instrumentalities and combinations particular point out in the appended claims.

According to the present invention, the foregoing and other objects and advantages are attained by a safety disposable injector comprising a barrel and a plunger slidably coupling within the barrel in a reciprocal manner.

In accordance with another aspect of the invention, the present invention comprises a safety disposable injector comprising a barrel having a chamber, and a plunger slidably coupled within the chamber of the barrel in a reciprocal manner, wherein the plunger has a tubular structure and comprises a plunger stopper provided at a top end of the plunger.

The safety disposable injector further comprises a medicament-dispensing needle unit and an injecting needle unit selectively coupled with the barrel in sealed manner.

The medicament-dispensing needle unit comprises a medicament-dispensing needle body and a medicament-dispensing needle base, wherein the medicament-dispensing needle body is upwardly extended from the medicament-dispensing needle base.

The injecting needle unit comprises an injecting needle body, an injecting needle base, a base sleeve, a sealing element, a compression spring, and a spring seat, wherein the base sleeve, which is a tubular member, comprises a retention protrusion protruded from an inner wall of the base sleeve at a bottom end thereof, wherein the sealing element is supported within the base sleeve by the retention protrusion so as to retain the sealing element at the bottom end of the base sleeve, wherein the injecting needle base is held within an interior cavity of the base sleeve, wherein the spring seat is coupled at a top end of the base sleeve, wherein the sealing element is sealed and retained between the bottom end of the base sleeve and the bottom end of the injecting needle base to provide a sealing effect therebetween and to apply a frictional holding force against the injecting needle base to withstand a compression force of the compression spring.

The safety disposable injector further comprises a sealer element, such as an O-shaped sealing ring, provided at the barrel to seal with the medicament-dispensing needle unit and the injecting needle unit when the medicament-dispensing needle unit or the injecting needle unit is coupled with the barrel.

A top opening end portion of the barrel has an inner diameter gradually reducing toward the bottom end thereof to form an inner conical opening at an inner surface of the barrel. Correspondingly, each of the medicament-dispensing needle unit and the injecting needle unit further has an outer conical platform formed at an outer circumferential surface of the bottom end of each of the medicament-dispensing needle unit and the injecting needle unit to seal and engage with the inner conical opening of the barrel.

The taper of the inner conical opening of the barrel is larger than the taper of each of the outer conical platform of the medicament-dispensing needle unit and the injecting needle unit.

The medicament-dispensing needle base further has a handling wing, a rhombus cross section (non-circular cross section), or knurled outer surface at the outer surface of the medicament-dispensing needle base for enabling the operator to hold and turn the medicament-dispensing needle base to attach to or detach from the barrel.

The injecting needle unit further comprises an injecting needle protection sleeve for covering the injecting needle unit, wherein the injecting needle protection sleeve is coupled with the base sleeve to enclose the injecting needle body within the injecting needle protection sleeve, wherein the injecting needle unit further comprises an inner tongue-groove structure provided at an inner surface of the injecting needle protection sleeve and an outer tongue-groove structure provided at an outer surface of the base sleeve to engage with the inner tongue-groove structure.

The injecting needle protection sleeve further has a handling wing, a rhombus cross section (non-circular cross section), or knurled outer surface for enabling the operator to hold and turn the injection needle unit to attach to the barrel.

The injecting needle unit further comprises an inner ratchet provided at an inner surface of the barrel and an outer ratchet provided at an outer surface of the base sleeve wherein when the base sleeve is rotatably coupled with the barrel, the inner ratchet is engaged with the outer ratchet.

The tip of the medicament-dispensing needle body is configured as one of a slanted tip and a conical tip with a side aperture at a circumferential surface of a tip portion.

The barrel, the plunger, the medicament-dispensing needle base, the injecting needle base, base sleeve, and the spring seat are made of plastic.

The medicament-dispensing needle body and the medicament-dispensing needle base of the medicament-dispensing needle unit are formed in a one piece integrated structure by mold injection.

The barrel is made of transparent plastic material.

Accordingly, the present invention provides the advantages as follows:

(1) According to the need of the injection, the operator is able to selectively interchange between the medicament-dispensing needle unit and the injecting needle unit to couple with the barrel. After the preparation of the liquid by the medicament-dispensing needle unit, the injecting needle unit to couple with the barrel to administer injections. Therefore, the sharpness of the injecting needle and the smoothness of the injecting needle will always be maintained at the best condition.

(2) The operation of the present invention is the same as the convention injection operation that the conventional operation habit remains basically unchanged in the present invention. The present invention is convenient to use and reliable to operate. After injection, the injector needle can be retracted by an automatic retraction mechanism to avoid the accidental hurt to medical staff and patients by a polluted needle tip.

(3) The present invention has a simple structural configuration and is suitable for automatic production on the assembly line, so that the present invention is capable of maintaining a high quality and reducing the manufacturing cost.

(4) As a result, the present invention will substantially reduce the unnecessary waste of material by interchanging the medicament-dispensing needle unit and the injecting needle unit, such that present invention is considered as an environmental friendly product.

Still further objects and advantages will become apparent from a consideration of the ensuing description and drawings.

These and other objectives, features, and advantages of the present invention will become apparent from the following detailed description, the accompanying drawings, and the appended claims.

### BRIEF DESCRIPTION OF THE DRAWINGS

FIG. 1 is a sectional view of a safety disposable injector with medicament-dispensing needle unit according to a preferred embodiment of the present invention, illustrating the wing-type medicament-dispensing needle unit.

FIG. 2 is a sectional view of the safety disposable injector with medicament-dispensing needle unit according to the above preferred embodiment of the present invention, illustrating the rhombus-type medicament-dispensing needle unit.

FIG. 3 is a sectional view of the safety disposable injector with injection needle unit according to the above preferred embodiment of the present invention.

FIG. 4 is a sectional view of the safety disposable injector with injection needle unit according to the above preferred embodiment of the present invention, illustrating the injection needled unit being retracted.

FIG. 5 is a sectional view of the injection needle unit of the safety disposable injector according to the above preferred embodiment of the present invention.

FIG. 6 is a sectional view of the medicament-dispensing needle unit of the safety disposable injector according to the above preferred embodiment of the present invention.

FIG. 7 is a sectional view of the injection needle unit of the safety disposable injector according to the above preferred embodiment of the present invention, illustrating the rhombus-type injection needle unit.

FIG. 8 is a sectional view of the medicament-dispensing needle unit of the safety disposable injector according to the above preferred embodiment of the present invention, illustrating the rhombus-type medicament-dispensing needle unit.

FIG. 9 illustrates a sealing structure of the barrel of the safety disposable injector with injection needle according to the above preferred embodiment of the present invention.

FIG. 10 illustrates an automatic retraction mechanism of the safety disposable injector with injection needle according to the above preferred embodiment of the present invention.

### DETAILED DESCRIPTION OF THE PREFERRED EMBODIMENT

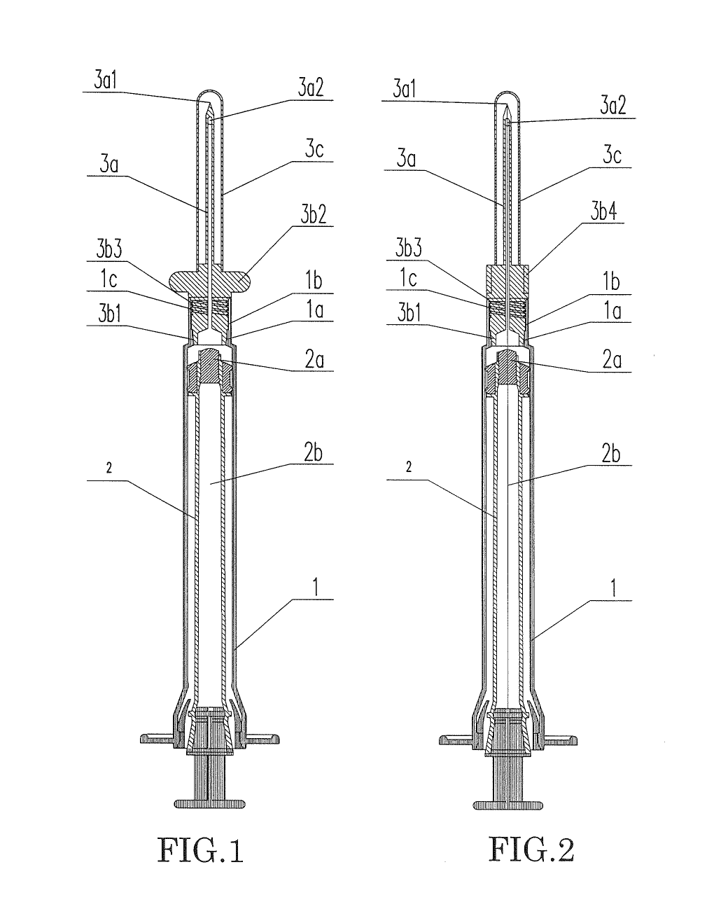

Referring to FIGS. 1 to 6 of the drawings, a safety disposable injector, such as a disposable syringe, according to a preferred embodiment of the present invention is illustrated, wherein the safety disposable injector comprises a barrel 1 having a chamber, and a plunger 2 slidably coupling within the chamber of the barrel 1 in a reciprocal manner. The safety disposable injector further comprises a medicament-dispensing needle unit 3 and an injecting needle unit 4 selectively coupling with the barrel 1. According to the preferred embodiment, the medicament-dispensing needle unit 3 comprises a medicament-dispensing needle body 3a and a medicament-dispensing needle base 3b, wherein the medicament-dispensing needle body 3a is fixed and upwardly extended from the medicament-dispensing needle base 3b. Alternatively, the medicament-dispensing needle body 3a and the medicament-dispensing needle base 3b of the medicament-dispensing needle unit 3 can be formed in a one piece integrated structure by mold injection. The injecting needle unit 4, which incorporates with an automatic retraction mechanism, comprises an injecting needle body 4a, an injecting needle base 4b, a base sleeve 4c, a sealing element 4f which is preferably a sealing O-ring, a compression spring 4d, and a spring seat 4e. The injecting needle body 4a is upwardly and coaxially extended from the injecting needle base 4b. The base sleeve 4c, which is a tubular member, comprises a retention protrusion 4c1 protruded from an inner wall of the base sleeve 4c at a bottom end thereof, wherein the sealing element 4f is supported within the base sleeve 4c by the retention protrusion 4c1 so as to retain the sealing element 4f at the bottom end of the base sleeve 4c. The injecting needle base 4b is held within an interior cavity of the base sleeve 4c. The spring seat 4e is coupled at a top end of the base sleeve 4c, wherein the spring seat 4e can be fixed to the base sleeve 4c by clamp-engagement, rotary-engagement, integrated structure or the like. The injecting needle body 4a is extended from the interior cavity of the base sleeve 4c to an exterior of the injecting needle base 4b through the spring seat 4e. The compression spring 4d is coaxially coupled with the injecting needle base 4b and is retained between the spring seat 4e and a bottom end of the injecting needle base 4b. Accordingly, the top end of the compression spring 4d is biased against the spring seat 4a while the bottom end of the compression spring 4d is biased against the bottom end of the injecting needle base 4b such that the compression spring 4d will apply a compression spring force against the injecting needle base 4b. In addition, the sealing element 4f is sealed and retained between the bottom end of the base sleeve 4c at the retention protrusion 4c1 thereof and the bottom end of the injecting needle base 4b to provide a sealing effect therebetween and to apply a frictional holding force against the injecting needle base 4b so as to withstand the compression force of the compression spring 4d. Accordingly, the frictional holding force of the sealing element 4f equals to or greater than the compression force of the compression spring 4d, such that the injection needle base 4b will be retained in position and the compression spring 4d is retained at a compressed state. In other words, the injection needle base 4b is held within the base sleeve 4c via the sealing element 4f. The barrel 1 has an inner threaded portion 1c. Correspondingly, the medicament-dispensing needle unit 3 further has an outer threaded portion 3b3 while the injecting needle unit 4 further has an outer threaded portion 4c5. According to the operation of the present invention, the medicament-dispensing needle unit 3 and the injecting needle unit 4 can be selectively coupled with the barrel 1 by rotatably engaging the outer threaded portion 3b3 of the medicament-dispensing needle unit 3 to the inner threaded portion 1c of the barrel 1 or by rotatably engaging the outer threaded portion 4c5 of the injecting needle unit 4 to the inner threaded portion 1c of the barrel 1. It is worth mentioning that the top opening end portion of the barrel 1 has an inner diameter gradually reducing toward the bottom end thereof to form an inner conical opening 1a at an inner surface of the barrel 1. Correspondingly, each of the medicament-dispensing needle unit 3 and the injecting needle unit 4 further has an outer conical platform 3b1, 4c3 formed at an outer circumferential surface of the bottom end of each of the medicament-dispensing needle unit 3 and the injecting needle unit 4. It is worth mentioning that each of the medicament-dispensing needle unit 3 and the injecting needle unit 4 can have a circular platform instead of the outer conical platform 3b1, 4c3. When the medicament-dispensing needle unit 3 or the injecting needle unit 4 is rotatably engaged with the barrel 1, the outer conical platform 3b1, 4c3 of the medicament-dispensing needle unit 3 or the injecting needle unit 4 will bias against the inner conical opening 1a of the barrel 1 so as to create a sealing effect between the outer conical platform 3b1, 4c3 of the medicament-dispensing needle unit 3/the injecting needle unit 4 and the inner conical opening 1a of the barrel 1. If the taper of the inner conical opening 1a of the barrel 1 is different from the taper of the outer conical platform 3b1, 4c3 of the medicament-dispensing needle unit 3 or the injecting needle unit 4, i.e. the taper of the inner conical opening 1a of the barrel 1 is larger than the taper of the outer conical platform 3b1, 4c3 of the medicament-dispensing needle unit 3 or the injecting needle unit 4, the sealing effect between the two surfaces will be achieved. For the best mode, when the taper of the inner conical opening 1a of the barrel 1 matches with the taper of the outer conical platform 3b1, 4c3 of the medicament-dispensing needle unit 3 or the injecting needle unit 4, the sealing effect between the surfaces will be maximized by increasing the contacting surface area. It is appreciated that an O-shaped sealing ring or other sealer element can be used for sealing between the two surfaces to further enhance the sealing effect therebetween. In order to operate the present invention, the medicament-dispensing needle unit 3 is firstly coupled with the barrel 1 such that by slidably pulling the plunger 2, the solvent is sucked into the barrel 1 via the medicament-dispensing needle unit 3. Then, by inserting the medicament-dispensing needle unit 3 into a bottle containing the dry powder medication, the solvent is injected into a bottle containing the dry powder medication via the slidably pushing the plunger 2, so as to dissolve the dry powder medication to form a liquid medication. By slidably pulling the plunger 2, the liquid medication is withdrawn from the bottle to the barrel 1 through the medicament-dispensing needle unit 3. The medicament-dispensing needle unit 3 is detached from the barrel 1 and the injection needle unit 4 is then coupled with the barrel 1. Accordingly, the injection needle unit 4 further has an outer ratchet 4c4 outwardly protruded from the outer circumferential surface thereof. Correspondingly, the barrel 1 further has an inner ratchet 1b protruded from the inner circumferential surface thereof, wherein when the injection needle unit 4 is rotatably engaged with the barrel 1 at one direction, the outer ratchet 4c4 is engaged with the inner ratchet 1b to prevent the injection needle unit 4 being reversely rotated back to an opposite direction. In other words, the injection needle unit 4 can only coupled with the barrel 1 but cannot detached from the barrel 1 once the injection needle unit 4 is coupled thereto. Once the injection needle unit 4 is coupled with the barrel 1, the plunger 2 is slidably pushed to administer injection through the injection needle unit 4. Accordingly, the plunger 2, having a tubular structure, comprises a plunger stopper 2a coupled at a top end of the plunger 2 and adapted to being pushed into the interior of the plunger 2 when a downward pushing force is applied at the plunger stopper 2a. When the plunger 2 is pushed toward the injection needle unit 4, the plunger stopper 2a of the plunger 2 will bias against the bottom end of the injection needle base 4b. When the pushing force is kept applying at the plunger 2, the plunger stopper 2a is forced and gradually pushed back into the interior of the plunger 2. At the mean time, the top end of the plunger 2 is aligned with the sealing element 4f and is slidably inserted into a gap between the bottom end of the base sleeve 4c and the bottom end of the injecting needle base 4b. When the pushing force is kept applying at the plunger 2, the top end of the plunger 2 will push against the sealing element 4f until the sealing element 4f is moved offset to its original position. Therefore, the frictional holding force of the sealing element 4f will be released to free the engagement between the base sleeve 4c and the injecting needle base 4b. It is worth mentioning that all the liquid medication will be discharged from the barrel 1 at this position. Once the frictional holding force of the sealing element 4f is released, the compression spring 4d will return back to its original form to apply the spring force against the injecting needle base 4b. Therefore, the injecting needle base 4b, including the injecting needle body 4a, will be pushed into the interior of the plunger 2 through the sealing element 4f so as to retract the injecting needle body 4a into the barrel 1. It is worth mentioning that the injecting needle base 4b will also push the plunger stopper 2a together into the interior of the plunger 2. In other words, when the injecting needle body 4a is rapidly retracted into the barrel 1, i.e. also the interior of the plunger 2, via the spring force, the injecting needle body 4a will be pulled back from the patient skin to complete the injection process. It is worth mentioning that the entire injecting needle body 4a is retracted into the interior of the plunger 2. Therefore, no protection cap is required for covering the top opening of the barrel to prevent the tip of the used injecting needle body 4a, so as to avoid an injury by accidental needle sticks. In addition, the used injection needle unit 4 cannot be re-assembled back to the barrel 1 to prevent the repeated use of the injection needle unit 4 for ensuring the injection needle unit 4 as a one-time use device.

### Embodiment 2

Embodiment 2 is a modification of the above embodiment, wherein the safety disposable injector further comprises a medicament-dispensing needle protection sleeve 3c for covering the medicament-dispensing needle unit 3. Accordingly, the medicament-dispensing needle protection sleeve 3c is coupled with the medicament-dispensing needle base 3b to enclose the medicament-dispensing needle body 3a within the medicament-dispensing needle protection sleeve 3c. The medicament-dispensing needle base 3b further has a handling wing 3b2, a rhombus cross section (non-circular cross section) 3b4, or knurled outer surface at the outer surface of the medicament-dispensing needle base 3b for enabling the operator to hold and turn the medicament-dispensing needle base 3b to attach to or detach from the barrel 1. Accordingly, the tip of the medicament-dispensing needle body 3a can be formed as a slanted tip or a conical tip 3a1 with a side aperture 3a2 at the circumferential surface of the tip portion.

### Embodiment 3

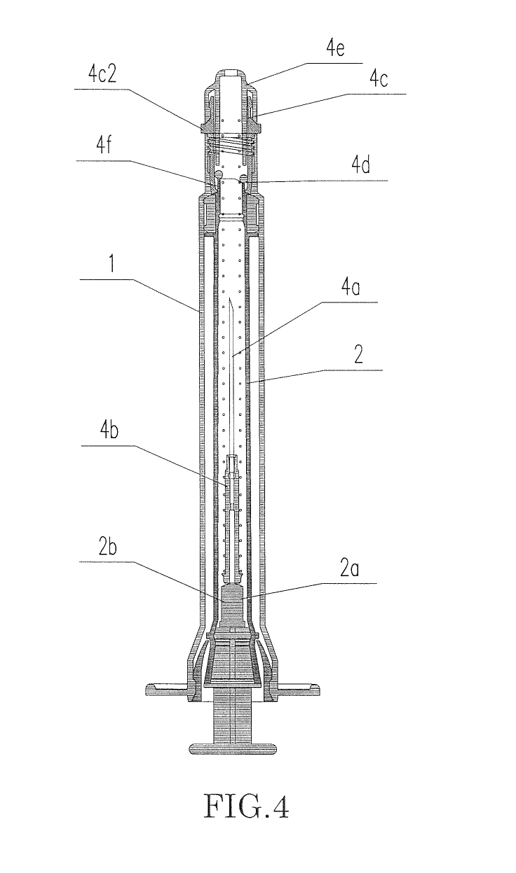

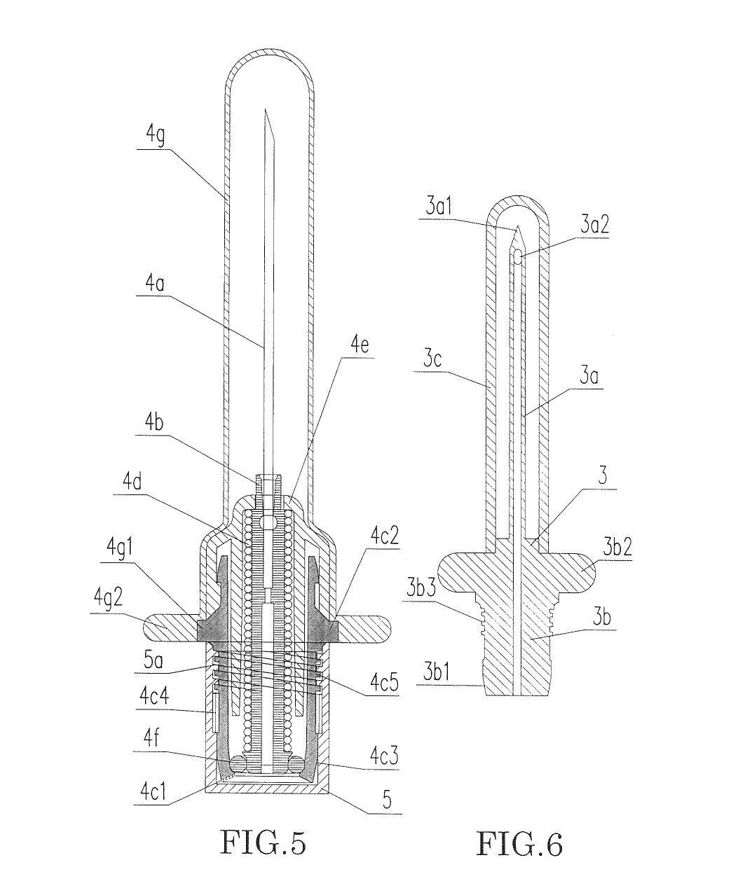

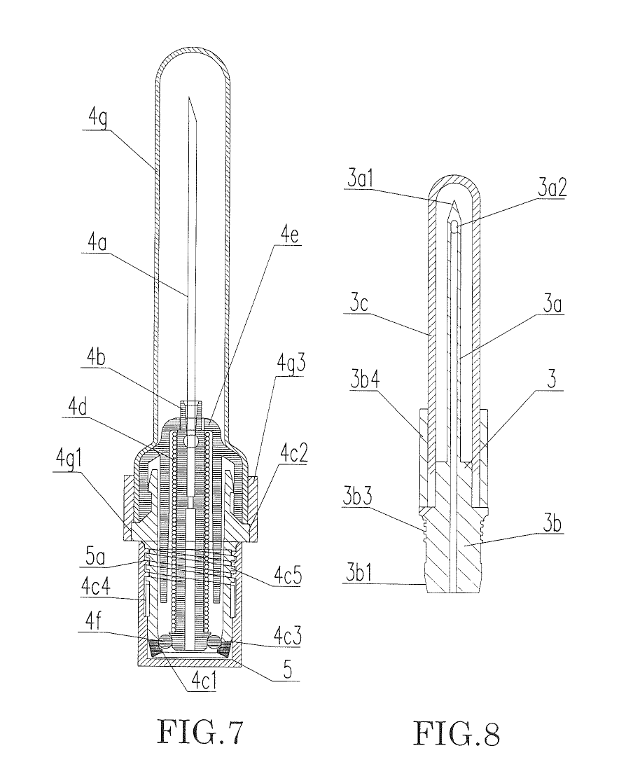

Embodiment 3 is another modification of the above embodiment, as shown in FIGS. 4 and 5, wherein the safety disposable injector further comprises an injecting needle protection sleeve 4g for covering the injecting needle unit 4. Accordingly, the injecting needle protection sleeve 4g is coupled with the base sleeve 4c to enclose the injecting needle body 4a within the injecting needle protection sleeve 4g. The injecting needle unit 4 further comprises an inner tongue-groove structure 4g1 provided at an inner surface of the injecting needle protection sleeve 4g and an outer tongue-groove structure 4c2 provided at an outer surface of the base sleeve 4c. Therefore, when the injecting needle protection sleeve 4g is coupled with the base sleeve 4c to enclose the injecting needle body 4a, the inner tongue-groove structure 4g1 is interlocked with the outer tongue-groove structure 4c2. The injecting needle protection sleeve 4g further has a handling wing 4g2, a rhombus cross section (non-circular cross section) 4g3, or knurled outer surface for enabling the operator to hold and turn the injection needle unit 4 to attach to the barrel 1. It is worth mentioning that before the injection needle unit 4 is attached to the barrel 1, the base sleeve 4c is covered and protected by a base cover 5. The base cover 5 can be removed from the base sleeve 4c in order for the injection needle unit 4 attaching to the barrel 1. Preferably, the base cover 5 has an inner threaded portion 5a rotatably and detachably coupled with the base sleeve 4c.

### Embodiment 4

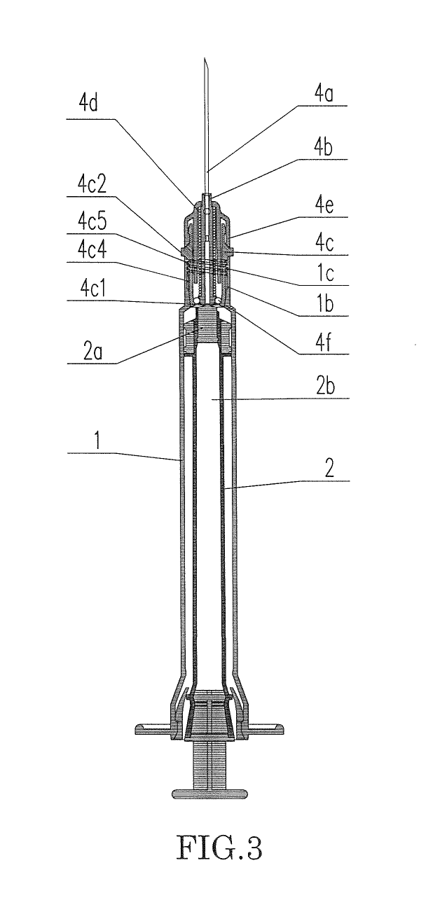

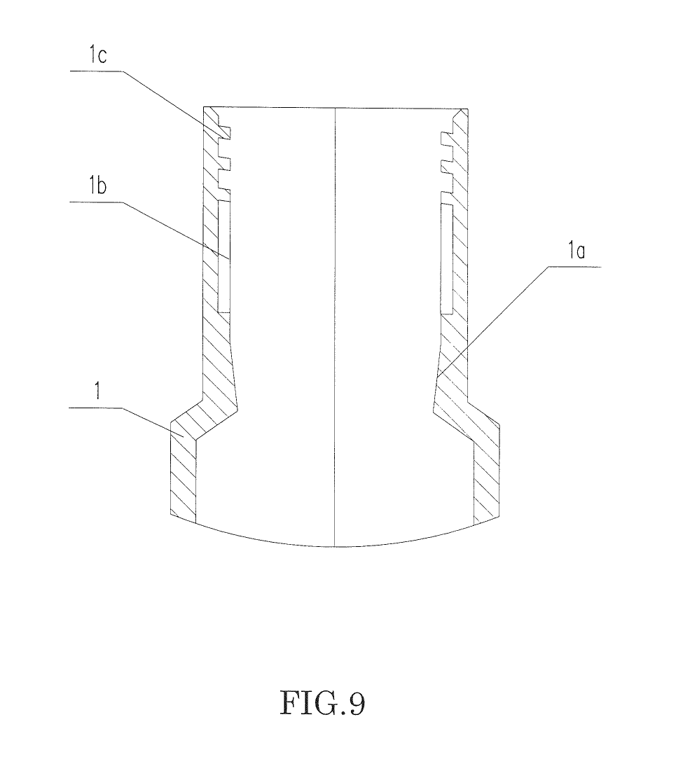

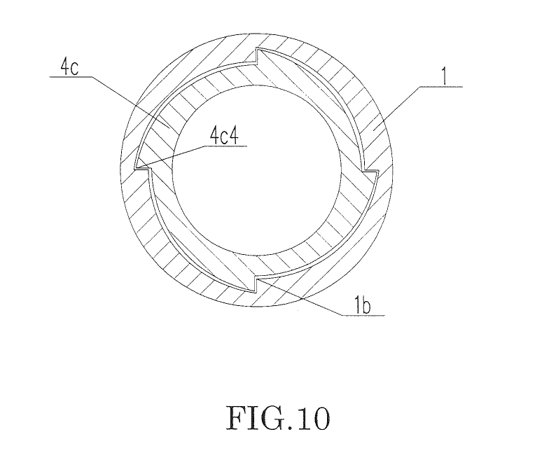

Embodiment 4 is another modification of the above first and third embodiments, as shown in FIGS. 3, 4, 7 and 9, wherein the automatic retraction mechanism of the injecting needle unit 4 further comprises an inner ratchet 1b provided at the inner surface of the barrel 1 and an outer ratchet 4c4 provided at the outer surface of the base sleeve 4c. When the base sleeve 4c is rotatably coupled with the barrel 1, the inner ratchet 1b is engaged with the outer ratchet 4c4. Therefore, the base sleeve 4c can only rotate at one direction to couple with the barrel 1, as shown in FIG. 10, so as to prevent the base sleeve 4c being detached from the barrel 1 by rotating the base sleeve 4c at the reverse direction. In other words, the present invention prevents the injecting needle unit 4 being re-used by disassembling the injecting needle unit 4 from the barrel 1 after use. Therefore, the present invention ensures the injecting needle unit 4 being used for one single time only.

One skilled in the art will understand that the embodiment of the present invention as shown in the drawings and described above is exemplary only and not intended to be limiting.

It will thus be seen that the objects of the present invention have been fully and effectively accomplished. It embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. A safety disposable injector, comprising:
a barrel 1 having a chamber,
a plunger 2 slidably coupled within said chamber of said barrel 1 in a reciprocal manner, wherein said plunger 2 has a tubular structure and comprises a plunger stopper 2a provided at a top end of said plunger 2;
a medicament-dispensing needle unit 3 comprising a medicament-dispensing needle body 3a and a medicament-dispensing needle base 3b, wherein said medicament-dispensing needle body 3a is upwardly extended from said medicament-dispensing needle base 3b; and
an injecting needle unit 4, wherein said medicament-dispensing needle unit 3 and said injecting needle unit 4 are selectively coupled with said barrel 1 in sealed manner;
wherein said injecting needle unit 4 comprises an injecting needle body 4a, an injecting needle base 4b, a base sleeve 4c, a sealing element 4f, a compression spring 4d, and a spring seat 4e, wherein said base sleeve 4c, which is a tubular member, comprises a retention protrusion 4c1 protruded from an inner wall of said base sleeve 4c at a bottom end thereof, wherein said sealing element 4f is supported within said base sleeve 4c by said retention protrusion 4c1 so as to retain said sealing element 4f at the bottom end of the base sleeve 4c, wherein said injecting needle base 4b is held within an interior cavity of said base sleeve 4c, wherein said spring seat 4e is coupled at a top end of said base sleeve 4c, wherein said sealing element 4f is sealed and retained between said bottom end of said base sleeve 4c and said bottom end of said injecting needle base 4b to provide a sealing effect therebetween and to apply a frictional holding force against said injecting needle base 4b to withstand a compression force of said compression spring 4d.

2. The safety disposable injector, as recited in claim 1, further comprising a sealer element provided at said barrel 1 to seal with said medicament-dispensing needle unit 3 and said injecting needle unit 4 when one of said medicament-dispensing needle unit 3 and said injecting needle unit 4 is coupled with said barrel 1.

3. The safety disposable injector, as recited in claim 1, wherein a top opening end portion of said barrel 1 has an inner diameter gradually reducing toward said bottom end thereof to form an inner conical opening 1a at an inner surface of said barrel 1, wherein, correspondingly, each of said medicament-dispensing needle unit 3 and said injecting needle unit 4 further has an outer conical platform 3b1, 4c3 formed at an outer circumferential surface of said bottom end of each of said medicament-dispensing needle unit 3 and said injecting needle unit 4 to seal and engage with said inner conical opening 1a of said barrel 1.

4. The safety disposable injector, as recited in claim 1, wherein said barrel 1 has an inner conical opening 1a, and correspondingly, each of said medicament-dispensing needle unit 3 and said injecting needle unit 4 further has an outer conical platform 3b1, 4c3, wherein a taper of said inner conical opening 1a of said barrel 1 is larger than a taper of each of said outer conical platform 3b1, 4c3 of said medicament-dispensing needle unit 3 and said injecting needle unit 4.

5. The safety disposable injector, as recited in claim 1, wherein said medicament-dispensing needle base 3b further comprises a means for enabling an operator to hold and turn said medicament-dispensing needle base 3b to attach to or detach from the barrel 1, wherein said means comprises a structure selected from the group consisting of a handling wing 3b2, a rhombus cross section 3b4, and knurled outer surface.

6. The safety disposable injector, as recited in claim 1, wherein said injecting needle unit 4 further comprises an injecting needle protection sleeve 4g for covering said injecting needle unit 4, wherein said injecting needle protection sleeve 4g is coupled with said base sleeve 4c to enclose said injecting needle body 4a within said injecting needle protection sleeve 4g, wherein said injecting needle unit 4 further comprises an inner tongue-groove structure 4g1 provided at an inner surface of said injecting needle protection sleeve 4g and an outer tongue-groove structure 4c2 provided at an outer surface of said base sleeve 4c to engage with said inner tongue-groove structure 4g1.

7. The safety disposable injector, as recited in claim 6, wherein said injecting needle protection sleeve 4g further comprises a means for enabling an operator to hold and turn said injection needle unit 4 to attach to said barrel 1, wherein said means comprises a structure selected from the group consisting of a handling wing 4g2, a rhombus cross section 4g3, and knurled outer surface.

8. The safety disposable injector, as recited in claim 1, wherein said injecting needle unit 4 further comprises an inner ratchet 1b provided at an inner surface of said barrel 1 and an outer ratchet 4c4 provided at an outer surface of the base sleeve 4c wherein when said base sleeve 4c is rotatably coupled with said barrel 1, said inner ratchet 1b is engaged with said outer ratchet 4c4.

9. The safety disposable injector, as recited in claim 1, wherein a tip of said medicament-dispensing needle body 3a is configured as one of a slanted tip and a conical tip 3a1 with a side aperture 3a2 at a circumferential surface of a tip portion.

10. The safety disposable injector, as recited in claim 1, wherein said barrel 1, said plunger 2, said medicament-dispensing needle base 3b, said injecting needle base 4b, base sleeve 4c, and said spring seat 4e are made of plastic.

11. The safety disposable injector, as recited in claim 1, wherein said medicament-dispensing needle body 3a and said medicament-dispensing needle base 3b of the medicament-dispensing needle unit 3 are formed in a one piece integrated structure by mold injection.

12. The safety disposable injector, as recited in claim 1, wherein said barrel 1 is made of transparent plastic material.
